(19) United States
(12) Patent Application Publication
(10) Pub. No.: US 2024/0237921 A1
(43) Pub. Date: Jul. 18, 2024

(54) AUTOMATED DIAGNOSTIC SYSTEM COMPRISING RODENTS

(71) Applicant: GILLUI SAVING LIVES LTD, Herzliya (IL)

(72) Inventors: Eran LUMBROSO, Herzliya (IL); Tal SHAUL, Givat Ada (IL); Israel ALVA, Karmei Yosef (IL)

(73) Assignee: GILLUI SAVING LIVES LTD, Herzliya (IL)

(21) Appl. No.: 18/561,608

(22) PCT Filed: May 17, 2022
(86) PCT No.: PCT/IL2022/050511

§ 371 Date: Nov. 16, 2023

(30) Foreign Application Priority Data
May 18, 2021 (IL) 283262

(51) Int. Cl. A61B 5/11; A01K 5/02

(52) U.S. Cl. CPC A61B 5/1113; A01K 5/02; A61B 2503/40; A61B 2503/42; A61B 2562/0247; A61B 2562/0257

(57) ABSTRACT

An automated diagnostic system and method for detection of a disease or disorder, comprising: a specialized device; rodents trained to perform a characteristic behavior pattern when exposed to a body fluid-derived test specimen from a subject having the disease or disorder; and a computerized sensing arrangement comprising sensors and a processor utility for receiving said sensed data, processing the sensed data to identify a disease-signifying event and outputting one or more event-related output signals comprising a diagnostic indication.

### TECHNOLOGICAL FIELD

The present disclosure relates to a system and a method for diagnosing a disease or disorder combining animals and computer-controlled sensing and recording elements.

### BACKGROUND OF THE INVENTION

Early detection is critical to successfully eradicate a variety of cancers. Existing technologies for early cancer detection are often costly, have a low accuracy, or may involve undesired procedures, such as patient's exposure to irradiation. Thus, techniques for early detection of diseases or disorder, in particular cancer are needed.

References considered to be relevant as background to the presently disclosed subject matter are briefly described below. Acknowledgement of the references herein is not to be inferred as meaning that these are in any way relevant to the patentability of the presently disclosed subject matter.

Webb et al, 2020, Behav Processes, 174:104085 Elsevier B. V., 174 Vol]: 104085 [pp.]), discloses that Since 1997, APOPO, a non-profit organization based in Tanzania, has deployed African giant pouched rats (Cricetomys ansorgei) to detect landmines in post-conflict areas.

U.S. Pat. No. 7,076,371 discloses a set of volatile markers which are characteristic of a particular condition or disease, and which are found in the exhaled breath of a person or odor from other parts of a body or from an entity. The detection is performed using a volatile substance detector, such as an artificial olfactory system.

US Patent Publication No. US 2017/0227491 discloses devices and methods to detect the presence of volatile organic compounds related to the presence of a disease state in a biological sample. The devices may include a detection moiety such as a polynucleotide in electronic communication with a semiconductor such as graphene or a carbon nanotube.

US Patent Publication No. US 2009/0145369 discloses a method and apparatus for detecting a target substance in a monitored environment using animals that perform an avoidance action upon detecting said target substance.

The main disadvantage of utilizing rat-based screening systems is that it is a labor-intensive process as it cannot be performed unattended (i.e., in the absence of an animals' trainer, laboratory staff, etc.). Therefore, there is a growing need to provide a new automated diagnostic system comprising rodents.

### SUMMARY OF THE INVENTION

Provided by this disclosure is an automated diagnostic system for detection of a disease or disorder that employs rodents, typically rats or mice, that are trained to sense specimens derived from individuals with a defined disease or disorder and behave in a certain characteristic behavioral pattern that may be detected by sensors that are coupled to a processor. The processor is configured to identify said behavioral pattern (that can be classified as a diseases-signifying event) and upon such identification output one or more output signals, comprising an indication that the specific specimen that caused the rodent to behave in said characteristic manner.

In the following the terms “rodent” or “rodents” will be used to denote one or a plurality of animals, respectively, that belong to the rodent order. Thus, the term rodent may refer, for example to a rat or a mouse and the term rodents may refer to two or more rats or mice. The term “rodents” may also refer to a plurality of animals of different species, for example one or more rats and one or more mice. Rat is a typical example of a rodent used in the device and method of this disclosure.

The term “disease or disorder” refers to any detectable abnormality. It may be a specific disease, a class of diseases or some other grouping of diseases or disorder. By some embodiments, the term disease or disorder may mean cancer in general, may be a class of cancers such as solid tumors, or may mean a specific cancer such as, but not limited to breast cancer, lung cancer, gastro-intestinal cancer, colorectal cancer, ovarian cancer, liver cancer, and pancreatic cancer. By other embodiments, the disease or disorder may be other classes of disease or disorder such as, for example, metabolic disease, neurological diseases, psychiatric disease, and others.

In the method or system of this disclosure, the one or more rodents serve as a diagnostic element. Rodents have a very sensitive sense of smell and this physiological characteristic is used to advantage for the purpose of medical diagnosis of a disease or disorder. Biological specimens from an individual having a disease or disorder including, for example cancer (e.g., solid tumor such as lung cancer), mental disorder and others (“diseased specimen(s)”), have a characteristic odor/smell that may be detected by rodents. Presumably, such biological specimens may include one or more volatile agents that may be different in a comparable specimen from normal individuals (“normal specimen(s)”, namely, individuals, not having the specific disease or disorder). The difference may be presence of such and agent that is absent in normal specimens, absence of such an agent that is present in a normal specimen, increase or decrease in the amount of such an agent, change in combination of such agents, etc. Consequently, the difference may cause changes (at time subtle) in odor between a normal specimen and a diseased specimen that may cause a different odor/smell that may be sensed by a rodent.

The specific smell of a diseased specimen, as compared to a normal specimen, will be referred to herein, at times, as “characteristic smell”.

The rodents used according to this disclosure are trained to perform a characteristic behavior pattern if exposed to a diseased specimen. This can be detected by the computer system and the specimen, the exposure to which causes the rodent to perform the characteristic behavior, can be earmarked as a suspected disease specimen. The specimens may be exposed, in a multiplexing procedure, to several rodents, all from the same or from a different species, and a repeated identification of a specimen as a suspected disease specimen may increase the confidence in the identification.

The specimen may be a body fluid such as urine, saliva, blood, lung aspirate, or mucus. The specimen may also be a concentrate, fraction or diluted form of said body fluid specimen sample.

The automated diagnostic system according to an embodiment of this disclosure, comprises a device, with one or more tracks defined therein, each configured to accommodate one or more rodents such that they can move freely in the one or more tracks. Each of the tracks has one or more openings in their floor and/or walls, e.g., of a diameter of about 20-30 mm.

This system also comprises one or more vessels, for accommodating a body-fluid derived test specimen, and one or more vessel holders for holding and positioning the vessels, each one below a separate opening.

The system also comprises one or more trained rodents, each of which being trained to perform a characteristic behavior pattern when exposed to a body fluid-derived test specimen from a subject having said disease or disorder (namely to a diseased specimen).

Comprised within said device are one or more sensors that are configured for sensing the activity of the one or more rodents that are accommodated within the device and moving within said track, particularly when the rodent passes over or comes into the vicinity of an opening. Such sensing causes the sensor to generate sensed data. The sensors are linked to a processor utility that is configured to receive the sensed data, process it to identify a disease-signifying event when said rodent performs said characteristic behavior pattern and outputting one or more event-related output signals in response to such identification. The output signal comprises a diagnostic indication that the specimen is suspected of being from a subject having said disease or disorder (namely a diseased specimen).

By an embodiment of this disclosure, the characteristic behavior pattern is sniffing at or into an opening for a time-period exceeding a predefined time, e.g., for a time exceeding 3 sec. Said characteristic behavior may comprise insertion of the rat's snout into the opening.

By one embodiment, each track is associated with a food dispenser configured for controlled dispensing of a food item; and said event-related output signal induces said controlled dispensing thereby rewarding the rodent for performing said characteristic behavior. Said dispenser may be in a dedicated feeding area reachable by the rodent from the one or more tracks through a controllable gate; and said one or more output signal may comprise opening said feeding area to permit entry of the rodent thereinto. By one exemplary configuration of the device, each track has or is associated with a rodent-launching cell separated from the track by a gate and said launching cell is also said dedicated feeding area.

Said one or more sensors may comprise one or more of optical sensor, motion sensor, touch sensor, pressure sensor, or proximity sensor. The one or more optical sensors may be an infra-red sensor.

By one embodiment said track is covered and is illuminated by an infra-red illumination system.

The processor utility is, typically, an artificial intelligence-based computing utility.

Said openings on the floor or walls of the track(s) or the vessels may comprise a controllable closure for selectively opening or closing the free air passage between the vessel and the track. The opening and closing of said closure are, typically, controlled by the processor utility, in correlation to movement or position of the rodent within the track. Thus, the opening of the closure may be typically timed to coincide with the rodent's approach to the respective opening and then close once the rodent distances from said opening. This may be important in order not to overwhelm or desensitize the rodent's olfactory system.

According to one embodiment of this disclosure, the device is configured for positioning of one specimen vessel beneath each opening. According to another embodiment, several specimen vessels may be placed below each opening and in turn each one may be brought into proximity to said opening.

Typically, as a quality control/assurance matter one or more of the vessels, which are fitted below the track's openings, contain a control specimen that may be a specimen derived from a body fluid sample of a diseased individual or from a pool of body fluid samples from a plurality of diseased individuals—a positive control; or may be a specimen derived from a body fluid sample of a healthy individual or from a pool of body fluid samples from a plurality of healthy individuals—a negative control. Each rodent may be randomly exposed in its track with said control specimen for a computerized assessment of accuracy of performance of said characteristic behavior pattern. Namely, the rodent's behavior may then be monitored when approaching these control specimens to see if the rodent reacts as expected (“properly”, namely ignores the negative controls and exhibits said characteristic behavior when confronting the positive controls). In the event the rodent does not react properly, the rodent's recorded responses with the test specimens may be ignored.

The device typically comprises one or more tracks, each one accommodating one rodent.

The vessels are typically held in a vessel holder configured for holding all vessels of a track. The device may also comprise a vessel holders' transporting system configured for transferring the holders between tracks for exposure of the specimens to different rodents. The diagnosis may then be based on analysis of the activity of different rodents in different tracks.

The system may comprise different groups of rodents, each one trained to detect one specific disease. In this way, each specimen may be subject to a plurality of diagnostic session (each session with a different group of rodents trained to detect different types of diseases).

The diagnostic method according to an embodiment of this disclosure comprises placing one or more rodents, each in a track with openings defined in its floor and/or walls, and permitting the rodents to move along the track, the rodents being trained to perform a characteristic behavior pattern when exposed to a body fluid-derived test specimen from a subject having said disease or disorder. Vessels are placed below said openings, each vessel containing a body fluid-derived test specimen. The behavior of each of the one or more rodents, while moving within the tracks, is monitored. This monitoring comprises: sensing activity of the one or more rodents while moving in said track through one or more sensors within the device that generate sensed data; and transferring the sensed data to a processor utility, configured for receiving said sensed data, processing the sensed data to identify a disease-signifying event when said rodent performs said characteristic behavior pattern at or in the vicinity of an opening, and outputting one or more event-related output signals in response to such identification, said output signals comprise a diagnostic indication that the specimen is suspected of being from a subject having said disease or disorder.

The present disclosure also provides a computerized training method for training rodents for use in the diagnostic method or system of this disclosure. This method has the purpose of training rodents to perform a characteristic behavior pattern when exposed to a body fluid-derived specimen from a subject having a certain disease or disorder. By this method one or more rodents are each placed in a track with openings that are defined in its floor and/or walls and the rodent is permitted to move along the track. Vessels are placed below these openings, the vessels containing control specimens that comprise (i) positive control specimens derived from one or more subjects having said disease or disorder, and (ii) negative control specimens derived from one or more subjects that do not have said disease or disorder. The rodent's behavior is monitored while they move within the track, the monitoring comprise (a) sensing activity of the one or more rodents while moving in said track through one or more sensors within the device that generate sensed data, (b) transferring the sensed data to a processor utility, configured for receiving said sensed data, processing the sensed data to identify a rodent that performs said characteristic behavior pattern at or in the vicinity of an opening, and (c) upon such identification, the processor utility generating an output signal that induces a reward sequence to the rodent.

The training method, by one embodiment, makes use of the device of the system described above.

The computerized training of rodents, as provided by this disclosure, can achieve predictable results, independent of a human trainer. Consequently, all rodents will have relatively uniform, expected, characteristic behavior pattern.

### DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic illustration of an exemplary automated diagnostic system for detection of a disease or disorder, in accordance with the presently disclosed subject matter;

FIG. 2 is a flowchart illustrating one example of a sequence of operations carried out by the system for detection of a disease or disorder, in accordance with the presently disclosed subject matter; and

FIG. 3 is a flowchart illustrating one example of a sequence of operations carried out by the system for training a rodent, in accordance with the presently disclosed subject matter.

### DETAILED DESCRIPTION OF THE INVENTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “monitoring”, “analyzing”, “generating”, “transferring”, “outputting”, “communicating”, “receiving” or the like, include action and/or processes of a computer that manipulate and/or transform data into other data, said data represented as physical quantities, e.g. such as electronic quantities, and/or said data representing the physical objects. The terms “computer”, “processor”, “processing circuitry” and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field programmable gate array (FPGA), an application specific integrated circuit (ASIC), etc.), a group of multiple physical machines sharing performance of various tasks, virtual servers co-residing on a single physical machine, any other electronic computing device, and/or any combination thereof.

The operations in accordance with the teachings herein may be performed by a computer specially constructed for the desired purposes or by a general-purpose computer specially configured for the desired purpose by a computer program stored in a non-transitory computer readable storage medium. The term “non-transitory” is used herein to exclude transitory, propagating signals, but to otherwise include any volatile or non-volatile computer memory technology suitable to the application.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

According to the presently disclosed subject matter, there is provided an automated diagnostic system for detection of a disease or disorder by utilizing one or more biosensors (e.g., rat or mice) that are trained to perform a characteristic behavior pattern when exposed to a specimen derived from a subject having the disease or disorder.

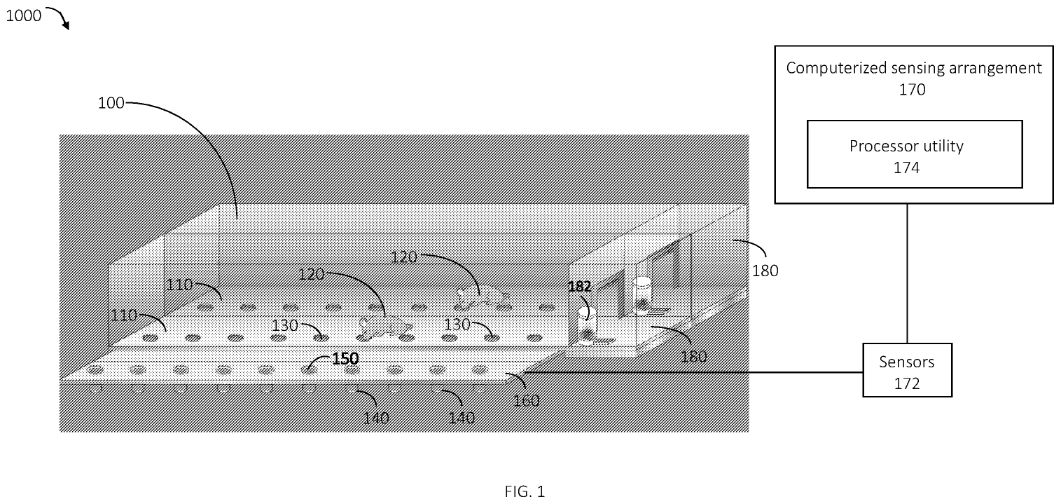

Bearing this in mind, attention is drawn to FIG. 1 that shows a schematic illustration of an exemplary automated diagnostic system 1000 for detection of a disease or disorder, in accordance with the presently disclosed subject matter.

The system 1000 includes a device 100 that includes one or more tracks 110. Each track 110 is configured to accommodate one rodent 120 (e.g., a rat or mouse) such that he can move freely thereon. In some cases, each track 110 has a rodent-launching cell 180 that is separated from the track 110 by a controllable gate. In addition, a plurality of openings 130 are defined in the floor of each track 110 so that the rodent 120 can pass above these openings 130 while moving along the track 110. As can be appreciated, by other embodiments, the openings may be defined, in addition or in the alternative, in one or more of the walls of the track. During rodent's movement along the track 110, each opening 130 is configured to open when the system recognizes that the rodent is in the vicinity of the opening, to expose him to a specimen located there beneath.

Rodents are highly sensitive to smells due to a unique structure of their olfactory system and therefore can be trained to perform a characteristic behavior pattern when exposed to certain smells of interest. For example, rodents can be trained to identify a diseased specimen that was derived from a diseased person. Such identification is demonstrated by characteristic behavior patterns of the rodent. In some cases, the characteristic behavior pattern may be an activity pattern at or in the vicinity of the opening 130. For example, the activity pattern may be sniffing the opening 130 (e.g., by insertion of the rat's snout into the opening) for a time-period exceeding a predefined time (e.g., for a duration of approximately 3 seconds).

Specimens can be body fluid-derived test specimens, such as for example but not limited to, body fluid samples selected from urine, saliva, blood, lung aspirate, mucus or concentrates, fraction or diluted form of said body fluid samples. Each opening 130 may have an aperture of a predetermined diameter (e.g., within the range of about 20 to about 30 mm for rats) that is suitable for rodent species that was chosen to perform inspection of the body fluid-derived test specimens.

The system 1000 further includes a plurality of vessels 140, wherein each vessel 140 is adapted to accommodate therein a body-fluid derived test specimen 150 from a subject that may have a disease or disorder, such as for example cancer (e.g., lung cancer). That is, the body-fluid derived test specimen may have been derived from a potentially diseased, diseased, healthy or of an unknown condition subject. Additionally, one or more vessel holders 160 are configured for holding and positioning the vessels 140, each one below a separate opening 130. In some cases, the one or more vessel holders 160 may be configured for holding all vessels for a track 110.

Device 100 further includes a computerized sensing arrangement 170 that is configured to monitor rodent's movement along the track and control various components of the device 100 accordingly. The computerized sensing arrangement 170 may include one or more sensors 172 that can be configured for sensing activity of the one or more rodents while they are moving along their respective tracks 110, e.g., when a rodent is passing over or coming into the vicinity of an opening 130 and generating sensed data. Sensors 172 may be, for example but not limited to, one or more of: an optical sensor, a motion sensor, a touch sensor, a pressure sensor, a proximity sensor or infrared sensor.

Computerized sensing arrangement 170 further includes a processor utility 174 configured to receive the sensed data obtained by sensors 172 and process the sensed data. Processor utility 174 may be one or more processing units (for example and without limitation, central processing units), microprocessors, microcontrollers (for example and without limitation, microcontroller units (MCUs)) or any other computing devices or modules, including multiple and/or parallel and/or distributed processing units, which are adapted to independently or cooperatively process data for controlling relevant management system 1000 resources and for enabling operations related to management system's 1000 resources. Processor utility 174 is configured to process the sensed data, obtained by sensors 172, to identify a disease-signifying event when a rodent performs the characteristic behavior pattern. For example, insertion of the rat's snout for a time period, above a certain predefine time, into the opening 130 may be identified as a disease-signifying event. Consequently, processor utility 174 may output one or more event-related output signals in response to such identification, wherein the output signals include a diagnostic indication that the specimen is suspected of being from a subject having a disease or disorder. In some cases, processor utility 174 is an artificial intelligence based (AI-based) computing utility.

Each track 110 of the device 100 may be associated with one or more of: a food dispenser 182 configured for controlled dispensing of a food item, a water dispenser configured for controlled dispensing of water or any other positive reinforcement and/or combinations thereof that can be provided to the rodent. In some cases, when a disease-signifying event is identified, event-related output signal that is generated in response to such identification, may induce a controlled dispensing of a food item and/or water to reward the rodent for performing a characteristic behavior pattern. For example, when a rodent performed an identification of a diseased specimen, he can be rewarded by receiving a food item and/or water, that may be provided by a respective dispenser located in a dedicated feeding area reachable by the rodent from the track 110 through a controllable gate. The state of the controllable gate (e.g., opened or closed) may be controlled by the processor utility 174 based on sensed data obtained by sensors 172 (e.g., readings from the motion sensor). In some cases, event-related output signal may include opening of the feeding area to permit entry of the rodent thereinto.

In some cases, rodent-launching cell 180 is also the dedicated feeding area.

In some cases, the food dispenser 182 and/or water dispenser may include a sensor configured to monitor head insertion of the rodent therein.

In some cases, openings 130 or vessels 140 contain a controllable closure for selectively opening or closing the free air passage between the vessel and the track 110. The opening and closing of the controllable closure may be controlled by the processor utility 174, for example in correlation to movement or position of the rodent 120 within the track 110. For instance, the vessel 140 may be a test tube with a folding leaves' closure that is configured to be opened by an elongated projection appended from the device and that inserts through the closure leaves to thereby open the vessel and allow air passage between the vessel and the respective opening 130 when the vessel is brought into proximity therewith. The leaves are urged towards a closed state and close spontaneously when the vessel is distanced from the opening.

In some cases, the vessels 140 contain a control specimen derived from a healthy or diseased individual, so that each rodent is randomly exposed in its track with said control specimen for a computerized assessment of accuracy of performance of his characteristic behavior pattern.

In some cases, device 100 further includes a vessel holders' transporting system that may be configured for transferring the vessel holders 160 between tracks 110 for exposure of the specimens to different rodents. This way, the same specimens may be inspected by different rodents thereby increasing the reliability of the rodents' performance. Additionally, the diagnostic indication that the specimen is suspected of being from a subject having a disease or disorder may be based on analysis, performed by processor utility 174, of the activity of different rodents in different tracks. It is to be noted that in some cases, the diagnostic indication may not be associated with a specific a disease or disorder rather a plurality of disease or disorder types. For example, the diagnostic indication may indicate that a specimen is suspected of being from a subject having cancer wherein the specific type of cancer should be further investigated.

In some cases, device 100 may be opaque to visible light and track 110 may be covered and lit using an infra-red illumination system so that the inspection procedure performed by the one or more rodents may be visible to an outside viewer via an infra-red camera for example. In other cases, the infra-red camera may monitor the rodent's activity within track 110 without the need for an auxiliary infra-red illumination. It is to be noted however that utilizing an infra-red camera is by no means limiting and the teachings herein can be performed utilizing any other thermographic imaging devices, mutatis mutandis.

### Exemplary Diagnostic Test Using the Device

According to one example of the presently disclosed subject matter, device 100 includes several test tracks 110 (e.g., 7 parallel tracks) each associated with a rodent-launching cell 180 comprising a computer-controlled automatic food and/or water dispenser 182 and a computer-controlled gate that separates between each rodent-launching cell 180 and its respective test track 110. Each track 110 includes multiple sniffing openings 130 (e.g., 10 openings) distributed evenly along the track, with computer-controllable closures associated therewith. Additionally, each track 110 further includes sensors for monitoring rodents' behavior while they are performing specimen discrimination test utilizing their olfactory system. For this purpose, seven trained rats are placed each in a separate rodent-launching cell 180. Urine samples are taken out from storage and transported in a computer-controlled automatic manner (e.g., by a robotic arm) to the first test track 110. That is, samples to be diagnosed are transported automatically from the storage area, each into an appropriate vessel 140 configured for accommodating such samples beneath respective openings 130 of track no. 1. Controllable gate of rodent-launching cell 180 associated with track no. 1 is opened and rat no. 1 exits therefrom onto the first test track 110. The controllable gate of the rodent-launching cell 180 automatically closes and the first sniffing opening 130, opens. The rat sniffs the air released from the sample by inserting its snout into the opening. A sensor detects the insertion of the rat's head into the opening (e.g., by monitoring a gyro sensor located on the rat) and measures the sniffing time. If the rat keeps the snout inside the opening for less than 3 seconds, a negative indication of disease is registered (i.e., no disease). The opening closes, and the next opening opens and the rat proceeds to sniff the next opening. If the rat keeps the snout inside the opening for a duration of 3 seconds or more, a positive indication of disease is registered (i.e., disease is detected). Optionally, upon such positive indication, a signal, which may be an audio signal, a visual signal, etc., may be displayed signaling the rodent of a reward awaiting at the launching cell 180. The rodent-launching cell 180 automatically opens (e.g., as a response to a signal received from the computer, e.g., processor utility 174) and the rat returns to the cell for receiving a positive reinforcement therein (as the rat is trained to behave accordingly). The opening and the controllable gate of the rodent-launching cell 180 are automatically closed. Upon expiration of approximately 20 seconds, rodent-launching cell 180 automatically reopens and rat no. 1 exits therefrom onto the first test track to complete her diagnostic test. Openings that were already scanned by the rat will remain closed while the first unscanned opening will automatically open. The rat will advance towards this opening and sniff the air released from the sample by inserting its snout into the opening. If the rat keeps the snout inside the opening for less than 3 seconds, a negative indication of disease is registered (i.e., no disease). The opening closes, and the next opening opens and the rat proceeds to sniff the next opening and so on until completion of examination of all the openings included in the track. After completion of sniffing all the openings in the track, the controllable gate of the rodent-launching cell 180 automatically opens and the rat returns to the launching cell. The controllable gate of the rodent-launching cell 180 automatically closes, and the computer (e.g., by utilizing the processor utility 174) sends a signal to the automatic food and/or water dispenser 182 and to dispense a positive reinforcement (e.g., by disposing a preferred food item, providing access to water, etc.) to the rat.

Upon completion of scanning all the openings (e.g., 10 openings) of the first track by rat no. 1, the samples of this track are automatically conveyed (e.g., by utilizing vessel holders' transporting system) to the next track (e.g., a neighboring track that may be denoted as track no. 2) to be examined by another rat (e.g., rat no. 2 respectively). The examination of the urine samples (e.g., urine samples that were tested in track no. 1 by rat no. 1 and automatically conveyed to track no. 2) in track no. 2 is performed in the same manner, by rat no. 2, as in track no. 1 by rat no. 1. Once the samples are evacuated from track no. 1, a new series of samples to be tested is automatically transported from the storage area to the appropriate vessels 140 of track no. 1, beneath respective openings 130 of the track.

This process is repeated (samples are transferred to track no. 3 and so on) until each series of samples is examined by all seven rats accommodated by the device 100.

The rats, each accommodated in a respective test track, repeatedly perform examination of the different series of samples for about one hour. One hour is considered a “working shift” and then the rats are removed from the device 100.

In some cases, the air can be pumped out of each track 110 upon completion of rodent's examination process of the samples and replaced with clean air in order to ventilate odors from previous samples and thereby enhance rodent's operation.

It is to be noted that all the operations of system 1000 disclosed herein are fully automated and may be performed in accordance with predetermined settings that may be adjusted to specific requirements and/or needs. These operations may be for example and without limitation, automatic transportation of the samples from the storage area to a respective track of device 100, automatic operation of the controllable gate, automatic operation of all openings 130 in accordance with rodent's movement along a respective track 110, monitoring each rodent's sniffing duration at a respective opening, monitoring and analyzing all other sensors' readings (e.g., readings from one or more of: an optical sensor, a motion sensor, a touch sensor, a pressure sensor, a proximity sensor, infrared sensor, etc.), automatic operation of the food and/or water dispensers, and/or any other operations that may be performed in accordance with the presently disclosed subject matter. Consequently, these operations may be performed unattended (i.e., in the absence of an animals' trainer, laboratory staff, system's operator, etc.) thereby obviating the need for operator's discretion and intervention during the examination process.

It is to be further noted that system 1000 is an automatic diagnostic system 1000 for detection of a disease or disorder wherein the diagnosis can be performed in a short time, non-invasively, without applying radiation to the subject and without hospitalization, thereby enabling, inter alia, a very convenient yearly screening process for high-risk populations. System 1000 may be configured to perform screening tests in the capacity of dozens, hundreds or more screening tests on a daily basis, wherein each test track 110 may be operated independently from all other tracks comprised by the device 100.

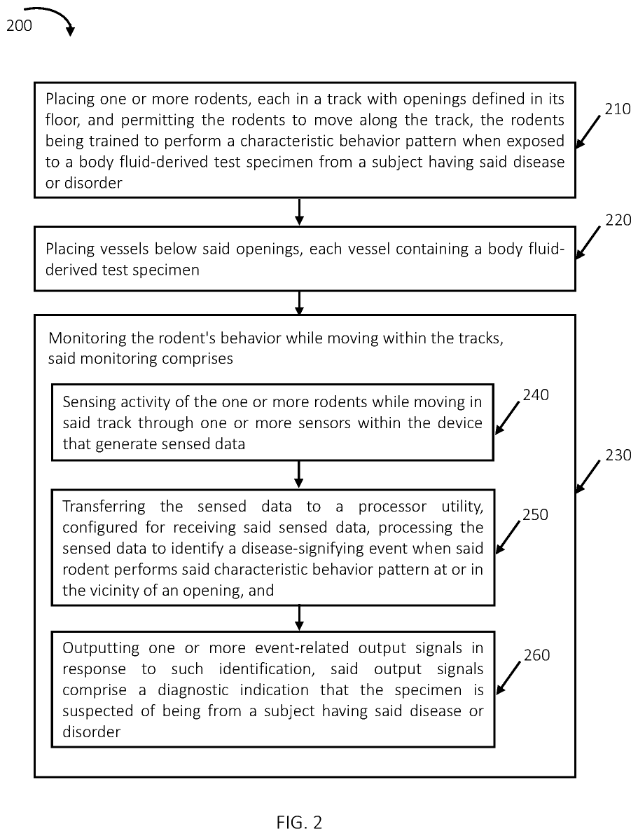

It is to be yet further noted that system 1000 is designed to support each rodent's safety. That is, it does not include grooves, sharp edges or moving parts therein that may harm or injure the rodent. Moreover, the system is very easy to clean wherein the structure thereof enables a convenient access for cleaning purposes even during operation. For this purpose, the system can include smooth surfaces made from non-absorbent materials, resistant to detergents and scratches. Attention is now drawn to FIG. 2, a flowchart illustrating one example of a sequence of operations carried out for detection of a disease or disorder process 200, in accordance with an embodiment of the presently disclosed subject matter.

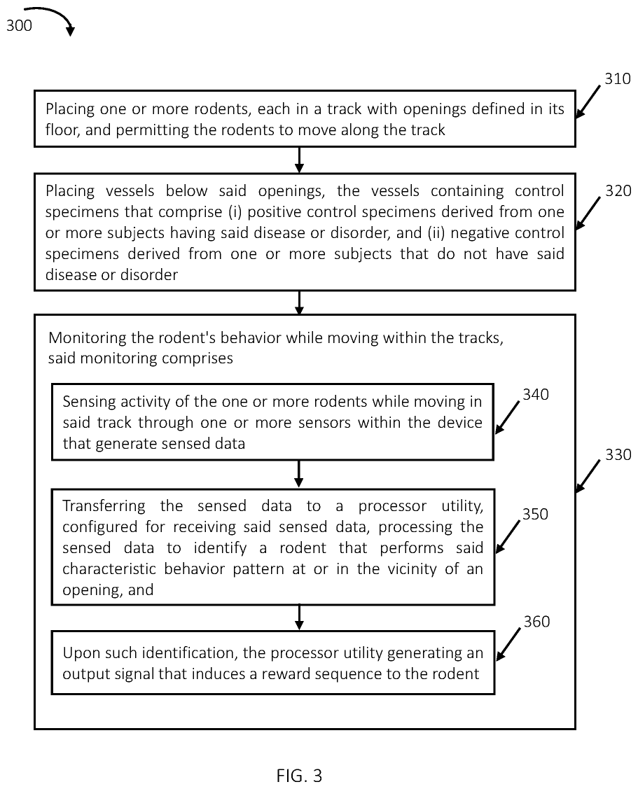

According to the presently disclosed subject matter, system 1000 can be configured to perform a detection of a disease or disorder, e.g., by process 200. The process 200 may start with step 210 wherein placing one or more rodents 120, each in a track 110 with openings 130 defined in its floor and permitting the rodents 120 to move along the track 110. For this purpose, each rodent 120 may be released from a respective lunching cell 180 that is associated with a given track 110. The rodent 120 advances along the track 110 while passing above each opening 130 located there beneath and examining (e.g., by his snout) a respective body fluid-derived test specimen accommodated therein. That is, the rodent examines each body fluid-derived test specimen that is accommodated by a respective vessel. The rodents are being trained (e.g., by process 300 as described herein below with respect to FIG. 3) to perform a characteristic behavior pattern when exposed to the body fluid-derived test specimen from a subject having a disease or disorder.

Step 210 may be followed by step 220 of placing vessels 140 below the openings 130, wherein each vessel 140 containing a body fluid-derived test specimen.

In step 230, each rodent's behavior is monitored while they are moving, each in his respective track. The monitoring may include one or more of the following steps:

In step 240, the activity of the one or more rodents is sensed while each is moving in a respective track through one or more sensors (e.g., one or more of: an optical sensor, a motion sensor, a touch sensor, a pressure sensor, a proximity sensor or infrared sensor) within the device 100 and generates sensed data therefrom.

Next, in step 250, the sensed data is transferred to a processor utility 170, which in turn is configured to receive the sensed data and process thereof to identify a disease-signifying event when the rodent performs the characteristic behavior pattern at or in the vicinity of a given opening 130. Step 250 may be followed by step 260 wherein the processor utility 170 is configured to output one or more event-related output signals in response to such identification. The output signals may include a diagnostic indication that the specimen associated with the given opening 130 is suspected of being from a subject having a disease or disorder, such as for example cancer (e.g., lung cancer).

In some cases, system 1000 can be configured to perform an automated quality control inspection at any stage of the examination process described herein (e.g., detection of a disease or disorder process 200). For this purpose, during the “working shift” of each rodent, at least one series of samples presented thereto may include a predetermined number of control samples, for which the diagnostic indication is known. For example, one series of samples that is presented to rat no. 1 may include two control samples, wherein one control sample derived from a diseased subject and the other derived from a healthy subject. The computerized sensing arrangement 170 will monitor the behavior of rat no. 1, inter alia, with respect to these two control samples to assess performance thereof. That is, the computerized sensing arrangement 170 will automatically check whether each rodent performs properly the characteristic behavior pattern at the vicinity of respective openings associated with the control samples, and thereby assure that the examination results of each rodent are reliable.

In some cases, performance assessment (e.g., provided by computerized sensing arrangement 170) may include providing a quality score to each rodent that have undergone the automated quality control inspection. Quality score may be considered for example during an overall summary of all the examination results (e.g., associated with a respective sample) performed by all rodents that were occupied during a respective “working shift” (e.g., to determine if there are test results performed by rodents that should be disqualified).

It is to be noted that, with reference to FIG. 2, some of the steps can be integrated into a consolidated step or can be broken down to a few steps and/or other steps may be added. Furthermore, in some cases, the steps can be performed in a different order than described herein (for example, step 220 can be performed before step 210, etc.). It is to be further noted that some of the steps are optional. It should be also noted that whilst the flow diagram is described also with reference to the system elements that realizes them, this is by no means binding, and the steps can be performed by elements other than those described herein.

Attention is now drawn to FIG. 3, a flowchart illustrating one example of a sequence of operations carried out for a rodent training process 300, in accordance with the presently disclosed subject matter.

According to an embodiment of the presently disclosed subject matter, system 1000 can be configured to perform a rodent training process 300. The process 300 may start with step 310 wherein placing one or more rodents, each in a track 110 with openings 130 defined in its floor and permitting the rodents to move along the track 110.

Next, in step 320, vessels 140 are placed below each opening 130 wherein each vessel is containing a control specimen, which may be one of the following:
(i) a positive control specimen derived from one or more subjects having a disease or disorder, such as for example cancer (e.g., lung cancer); or
(ii) a negative control specimen derived from one or more subjects that do not have a disease or disorder, such as for example cancer (e.g., lung cancer).

For the rodents to reliably recognize an odor, behavioral training involving positive reinforcement is required. For example, in order to train the rodent to recognize an odor of a diseased specimen, the rodent is required to travel along track 110 and be exposed (e.g., via openings 130) to control specimens, comprising at least one positive control specimen. Upon performing a characteristic behavior pattern by the rodent (e.g., performing an activity pattern at or in the vicinity of opening 130, such as for example, insertion of the rodent's snout into the opening, in some cases for a time-period exceeding a predefined time, e.g., for a duration of or exceeding approximately 3 seconds) when it identifies the positive control specimen, positive reinforcement is provided to the rodent (e.g., by disposing a preferred food item, providing access to water, etc.). Step 320 may be followed by step 330 of monitoring the rodent's behavior while it is moving within the track 110. The monitoring may include one or more of the following steps:

In step 340, the activity of the rodent is sensed while it is moving in a respective track through one or more sensors (e.g., one or more of: an optical sensor, a motion sensor, a touch sensor, a pressure sensor, a proximity sensor or infrared sensor) within the device 100 and generates sensed data therefrom.

Next, in step 350, the sensed data is transferred to the processor utility 170, which in turn is configured to receive the sensed data and process thereof to identify a rodent that performs the characteristic behavior pattern at or in the vicinity of an opening of interest (e.g., an opening that is associated with the positive control specimen).

Process 300 may end at step 360 in which upon such identification (e.g., an identification of one or more rodents that performed the desired characteristic behavior pattern), the processor utility 170 can be configured to generate an output signal that induces a reward sequence to the rodent. The reward sequence may be for example opening a controllable gate to permit entry of the rodent into the feeding area, wherein a preferred food item and/or water may be disposed to reward the rodent. In some cases, the feeding area may be also the rodent-launching cell 180.

In accordance with the presently disclosed subject matter, system 1000 can be utilized to perform an automated rodent training process (e.g., rodent training process 300 disclosed herein), to foster each rodent from a naive state (e.g., an untrained state) to an independent operative state (e.g., a fully trained state wherein the rodent is able to perform independently a detection of a disease or disorder process 200). This can be achieved by exposing each rodent to control samples, for which the diagnostic indication is known, and monitoring and analyzing their behavior is response to such exposure. The system 1000 is configured to automatically respond in accordance with each rodent's behavior. For example, if at a given stage of the training process the rodent is expected to perform a characteristic behavior pattern when exposed to a positive control specimen (e.g., a body fluid-derived specimen from a subject having a certain disease or disorder, such as but not limited to, lung cancer), computerized sensing arrangement 170 may be configured to verify if the rodent indeed performed the expected characteristic behavior pattern, and if so the computerized sensing arrangement 170 may perform series of actions, such as for example opening the controllable gate of the rodent-launching cell 180 and automatically dispensing a preferred food item and/or water to encourage the rodent to return to the rodent-launching cell 180 for receiving the positive reinforcement therein. This way, the rodent may learn to immediately return to the rodent-launching cell 180 upon performing the characteristic behavior pattern.

Each stage of the rodent training process may be performed in accordance with predetermined steps and protocols, that may be adjusted to specific requirements and/needs (e.g., the type of the animal that is being trained). In addition, system 1000 can be configured to determine if the rodent is ready to proceed to the next stage of the training process if rodent's performance exceeds a predetermined threshold. For example, one stage of the training process may be detecting one positive control specimen associated with a specific disease while the next stage thereof may be detecting two positive control specimens associated with two different diseases, each having a unique odor associated therewith. Final stage of the training process may be a “final test” wherein each rodent is required to perform full specimen examination process (e.g., detection of a disease or disorder process 200).

It is to be noted that the automated rodent training process of system 1000 can be performed unattended (i.e., in the absence of an animals' trainer, laboratory staff, system's operator, etc.) thereby obviating the need for operator's discretion and intervention during the training process. It is to be noted that, with reference to FIG. 3, some of the steps can be integrated into a consolidated step or can be broken down to a few steps and/or other steps may be added. Furthermore, in some cases, the steps can be performed in a different order than described herein (for example, step 320 can be performed before step 310, etc.). It is to be further noted that some of the steps are optional. It should be also noted that whilst the flow diagram is described also with reference to the system elements that realizes them, this is by no means binding, and the steps can be performed by elements other than those described herein.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

1.-50. (canceled)

51. An automated diagnostic system for detection of a disease or disorder, comprising:
a device that comprises one or more tracks and configured to accommodate one or more rodents such that they can move freely in the one or more tracks;
one or more openings defined in one or more of the floor or walls of said tracks;
one or more vessels, for accommodating a body-fluid derived test specimen;
one or more vessel holders for holding and positioning the vessels, each one below a separate opening; and
a computerized sensing arrangement comprising:
(i) one or more sensors configured for sensing activity of the one or more rodents accommodated in said device when passing over or coming into the vicinity of the opening and generating sensed data, and
(ii) a processor utility for receiving said sensed data, processing the sensed data to identify a disease-signifying event when said rodent performs said characteristic behavior pattern and outputting one or more event-related output signals in response to such identification, said output signals comprise a diagnostic indication that the specimen is suspected of being from a subject having said disease or disorder
wherein the automated diagnostic system is configured to accommodate one or more trained rodents, each of which being trained to perform a characteristic behavior pattern when exposed to a body fluid-derived test specimen from a subject having the disease or disorder.

52. The diagnostic system of claim 51, wherein
each track is associated with a food dispenser configured for controlled dispensing of a food item; and
said event-related output signal induces said controlled dispensing thereby rewarding the rodent for performing said characteristic behavior pattern.

53. The diagnostic system of claim 52, wherein
said dispenser is in a dedicated feeding area reachable by the rodent from the one or more tracks through a controllable gate;
said one or more output signal comprise opening said feeding area to permit entry of the rodent thereinto.

54. The diagnostic system of claim 51, wherein said one or more sensors comprise one or more of an optical sensor, a motion sensor, a touch sensor, a pressure sensor, or a proximity sensor.

55. The diagnostic system of claim 51, wherein the processor utility is an artificial intelligence-based computing utility.

56. The diagnostic system of claim 51, wherein
said openings or vessels contain a controllable closure for selectively opening or closing the free air passage between the vessel and the track; and wherein
the opening and closing of said closure are controlled by said processor utility.

57. The diagnostic system of claim 51, wherein the opening and closing of said closure is controlled by said processor utility in correlation to movement or position of the rodent within the track.

58. The diagnostic system of claim 51, wherein
one or more of the vessels contain a control specimen derived from a healthy or diseased individual, and wherein
each rodent is randomly exposed in its track with said control specimen for a computerized assessment of accuracy of performance of said characteristic behavior pattern.

59. The diagnostic system of claim 51, wherein said one of more vessels holders are configured for holding all vessels for a track.

60. The diagnostic system of claim 59, comprising a vessel holders' transporting system configured for transferring the holders between tracks for exposure of the specimens to different rodents, and wherein said diagnostic indication is based on analysis of the activity of different rodents in different tracks.

61. A method for detection of a disease or disorder, comprising:
placing one or more rodents, each in a track with openings defined in one or more of its floor or walls, and permitting the rodents to move along the track, the rodents being trained to perform a characteristic behavior pattern when exposed to a body fluid-derived test specimen from a subject having said disease or disorder;
placing vessels below said openings, each vessel containing a body fluid-derived test specimen;
monitoring the rodent's behavior while moving within the tracks, said monitoring comprises sensing activity of the one or more rodents while moving in said track through one or more sensors within the device that generate sensed data,
transferring the sensed data to a processor utility, configured for receiving said sensed data, processing the sensed data to identify a disease-signifying event when said rodent performs said characteristic behavior pattern at or in the vicinity of an opening, and outputting one or more event-related output signals in response to such identification, said output signals comprise a diagnostic indication that the specimen is suspected of being from a subject having said disease or disorder.

62. The method of claim 61, wherein
each track is associated with a food dispenser configured for controlled dispensing of a food item; and
said event-related output signal induces said controlled dispensing to thereby reward the rodent for performing said characteristic behavior pattern.

63. The method of claim 61, wherein
said dispenser is in a dedicated feeding area reachable by the rodent from the one or more tracks through a controllable gate;
said one or more output signal comprise opening said gate to permit entry of the rodent into the feeding area.

64. The method of claim 61, wherein said one or more sensors comprise one or more of an optical sensor, a motion sensor, a touch sensor, a pressure sensor, or a proximity sensor.

65. The method of claim 61, wherein
said openings or vessels contain a controllable closure for selectively opening or closing the free air passage between the vessel and the track; and wherein
the opening and closing of said closure are controlled by said processor utility.

66. The method of claim 61, wherein said processor utility induces the opening and closing of said closure in correlation to movement or position of the rodent within the track.

67. The method of claim 61, comprising
placing one or more control vessels containing control specimens below,
monitoring activity of the rodents when in the vicinity of an opening associated with a control specimen, and comprising
determining, based on the monitored activity accuracy of the performance of said characteristic behavior pattern.

68. The method of claim 61, comprising
placing a plurality of rodents, each one in a separate track,
placing specimen-containing vessels below the opening of each track, and
monitoring the behavior of all rodents while moving within the tracks.

69. The method of claim 68, comprising transporting the vessels from one track to the other and outputting a diagnostic indication on the basis of analysis of the activity of different rodents in different tracks.

70. A method for training a rodent to perform a characteristic behavior pattern when exposed to a body fluid-derived specimen from a subject having a certain disease or disorder, the method comprising:
placing one or more rodents, each in a track with openings defined in one or more of its floor or walls, and permitting the rodents to move along the track;
placing vessels below said openings, the vessels containing control specimens that comprise (i) positive control specimens derived from one or more subjects having said disease or disorder, and (ii) negative control specimens derived from one or more subjects that do not have said disease or disorder;
monitoring the rodent's behavior while moving within the tracks, said monitoring comprises
sensing activity of the one or more rodents while moving in said track through one or more sensors within the device that generate sensed data,
transferring the sensed data to a processor utility, configured for receiving said sensed data, processing the sensed data to identify a rodent that performs said characteristic behavior pattern at or in the vicinity of an opening, and
upon such identification, the processor utility generating an output signal that induces a reward sequence to the rodent.
